(19) United States
(12) Patent Application Publication
(10) Pub. No.: US 2018/0353671 A1
(43) Pub. Date: Dec. 13, 2018

(54) DIALYSIS MACHINE

(71) Applicant: FRESENIUS MEDICAL CARE DEUTSCHLAND GMBH, Bad Homburg (DE)

(72) Inventor: Bernd TESSENDORF, Bad Homburg (DE)

(73) Assignee: FRESENIUS MEDICAL CARE DEUTSCHLAND GMBH, Bad Homburg (DE)

(21) Appl. No.: 15/781,222

(22) PCT Filed: Dec. 2, 2016
(86) PCT No.: PCT/EP2016/002036

§ 371 Date: Jun. 4, 2018

(30) Foreign Application Priority Data
Dec. 3, 2015 (DE) 10 2015 015 624.1

(51) Int. Cl. A61M 1/28; A61B 5/00; A61M 1/36; A61J 1/10

(52) U.S. Cl. CPC A61M 1/285; A61B 5/4836; A61B 5/14532; A61J 1/10; A61M 1/3659

(57) ABSTRACT

A dialysis machine, in particular a peritoneal dialysis machine, with one or more of the following components: at least one receptacle, in particular a bag, to receive fresh or used dialysate, at least one tubing set to conduct a liquid, in particular dialysate, at least one patient catheter for the uptake and/or discharge of dialysate into or from the patient's abdominal cavity, whereby at least one of the components is provided with at least one sensor designed to measure at least one parameter value of the dialysate and which is also designed to transmit the parameter value to at least one receiver.

The present invention relates to a dialysis machine, in particular a peritoneal dialysis machine, with one or more of the following components: at least one receptacle, in particular at least one bag, to receive fresh or used dialysate, at least one tubing set for conducting a fluid, in particular dialysate, and/or least one patient catheter for the inflow and outflow of dialysate into or from the patient's abdominal cavity.

Dialysis machines known from the state of the art are commonly operated on the basis of a so-called prescription determined by the physician prior to treatment. Thus, treatment is usually “static”, i.e. conducted according to a certain protocol. This can have the effect that treatment is possibly not optimal in terms of the patient's treatment efficiency or treatment comfort. In particular, the concentration of dialysate, the dwell time in the abdomen, the number of treatment cycles, and the inflow/outflow speed are fixed in the course of the treatment, i.e. they are set according to a predetermined prescription protocol.

Furthermore, in peritoneal dialysis machines known from the state of the art, the dialysate which is discharged by means of the patient catheter leading into the abdominal cavity, is usually not analyzed but disposed of. Tests are conducted only at irregular intervals and when it is necessary to determine the functional efficiency of the peritoneum. As a rule, these tests are laboriously conducted at the physician's office.

From EP 0 772 693 B1, a process and a mechanism is known to measure the concentration of a substance such as urea in a dialysate. The sensors employed in this are, for example, conductivity measurement cells. EP 0 711 182 B1 also describes the determination of urea in dialysis treatment whereby the fluid sample is, for example, passed by a sensor which is a component of a sampling unit of the dialysis machine. From EP 0 942 759 B1, a dialysis machine with a urea monitor is known that serves to monitor the dialysis efficiency and to adjust the type of treatment automatically.

WO 2013/170219 A1 discloses a monitoring system with a sensor implanted under the skin of the patient, whereby the measuring data of the sensor can be optically read. From EO 0 782 460 B1 a mechanism is known to conduct a so-called peritoneal equilibration test, whereby a number of metabolic breakdown products are determined to indicate the transport characteristics of the peritoneum and thus to establish a patient-specific peritoneal dialysis prescription.

Finally, WO 2015/012990 A1 describes how a peritoneal dialysis machine forwards alarm signals, for example to a mobile telephone.

Object of the present invention is to further develop a dialysis machine, in particular a peritoneal dialysis machine of the kind mentioned above to enable it to monitor treatment with especially simple means and if need be to adjust the treatment parameters.

This object is achieved by means of a dialysis machine with the characteristics of claim 1.

It is provided that at least one of the components, i.e. at least the receptacle and/or the tubing set and/or the patient catheter is provided with at least one sensor designed to measure at least one parameter value of the fluid inside the component, and which is also designed to transmit the parameter value to at least one receiver. The sensor can be designed such that the parameter value is measured in a liquid at rest or in a flowing liquid.

The present invention is therefore based on the idea to provide the sensor technology as part of the at least one component. The one or more sensors are arranged on or in the bag or on or in the patient catheter or on or in the tubing set and are thus not an integral part of the dialysis machine, which therefore can be of simpler design than known machines which have such sensor technology.

It is therefore the essential idea of the invention to design the at least one sensor as a part of one or more of the said components.

Preferably that component is a disposable article. i.e. a disposable, which means that the sensor is disposed of as well.

The present invention makes it possible to monitor the treatment in a simple manner, for example to provide the patient with the information whether the treatment being conducted is successful, or to provide the physician with the information whether the treatment has been successfully concluded or is proceeding successfully, to enable the physician to adjust the treatment parameters and, for example, to prescribe a more effective or gentler treatment. For example, the latter could consist of reducing the patient's glucose level caused by the dialysate and such to achieve a more tolerable and perhaps more efficient therapy.

This intervention or change of prescription can be done after a treatment or also preferably during a treatment, and in particular preferably in real time. It means that the physician or also the patient can if need be affect the treatment and change one or more of the treatment parameters.

For example, information can be collected to evaluate the treatment success, i.e. how much toxin is contained in the dialysate discharged from the abdominal cavity, a statement can be made about the functionality of the peritoneum, and in particular a statement can be made about the early recognition that the ultrafiltration rate is insufficient, and the early recognition of peritonitis is possible as well.

It is therefore conceivable to conduct a real time adjustment of the treatment parameters based on one or more of the sensor data.

It is particularly advantageous when the sensor is designed such that it can wirelessly transmit the at least one parameter value to at least one receiver.

The sensor can be designed such that it is possible to continuously measure the at least one parameter value and/or to transmit it such that a continuous monitoring of the treatment or a continuous recording of the treatment is possible.

The sensor can be designed and arranged such that that the fluid flows over it and/or through it.

The fluid can be any fluid that is used in the course of dialysis treatment or plays a role in it, such as the patient's blood, but in particular it is the dialysate itself.

The at least one sensor can be integrated in the wall of the component such as the bag, or it can be arranged inside or outside on the component. The significant factor is that the sensor is not an external part of the component, but is provided on it or inside it.

In another embodiment of the invention, it is provided that the at least one sensor is designed as a passive transponder or as an RFID chip or also as an active transmitter, i.e. as an active transmitter equipped with an energy source or also an active transponder.

Data transmission from the sensor to a receiver can take place in any way whatever, also wired, but wireless transmission is preferred. Transmission via RFID, ZigBee, Bluetooth, etc. is conceivable.

If the sensor is designed as a passive transponder, i.e. if it does not have its own power supply, a particularly cost-effective design of the component in question is possible.

In a preferred embodiment of the invention, the tubing set consists of at least two sections in fluid connection with a connector such as a break connector. The sensor can be provided at that connector such that the sensor is in connection with the two separate sections and is preferably arranged between these.

It is thus possible for example in peritoneal dialysis to provide the sensor in the line leading to the drainage bag. In this, the sensor can be arranged between a first tube section and between a second tube section that is in fluid communication with the drainage bag, whereby the second tube section can be the tube section of the tubing set which in the course of the previous treatment or a previous cycle was in connection with the receptacle(s) or bag(s) containing the fresh dialysate.

The at least one parameter can be, for example, the temperature, the pressure, the concentration of one or more metabolic breakdown products (such as urea, creatinine and electrolytes), the glucose level, the protein level, the turbidity, the conductivity of the fluid, i.e. in particular the dialysate, or also of the blood.

This list is not a complete but contains only some examples. In principle, the invention also includes any parameter whatsoever that can be measured by a sensor.

The dialysis machine can have at least one receiver capable of receiving the parameter(s) determined by the one or more sensors of the components. Furthermore, the dialysis machine can comprise at least one display unit and/or at least one processing unit, whereby the display unit can display or otherwise communicate the parameter, for example also acoustically.

The processing unit can be designed to control or regulate the operation of the dialysis machine depending on the one or more parameter values.

It is preferably provided that the control or regulation of the dialysis machine occurs in real time.

For example, the processing of the data such as the level of toxins or metabolic breakdown products in the fluid contained in the drainage bag of a peritoneal dialysis machine gives an indication to the physician who may introduce more accurate follow-up examinations and/or real time adjustment of the peritoneal dialysis parameters such as the dwell time of the fluid in the abdominal cavity. This allows an optimal real time-regulated peritoneal dialysis treatment.

As mentioned above, the one or more components are preferably disposables, such that the sensors are also disposed of after treatment of the patient. This always ensures sterile handling.

Furthermore, the dialysis machine can be designed such that its operation is controlled or regulated not only depending on the data generated by the sensors of the one or more components, but also by external sensors, i.e. sensors that are not part of the components in question (bags or other receptacles, tubing set, catheter). Thus treatment can also take into account data from other sensors such as data generated by a mobile telephone or sensors worn on the body. The data can also be generated by external devices such as weight scales or blood pressure monitors, etc.

Thus treatment can, for example, also be controlled or regulated depending on bio parameters such as the heart rate, the body temperature, etc.

The one or more receivers to which the data of the one or more sensors are transmitted are preferably components of the dialysis machine.

In a further embodiment of the invention it is provided that the one or more receivers are not components of the dialysis machine but at least one external device, preferably a mobile terminal device such as a smartphone or tablet.

It is thus possible to preferably continuously record the data measured by the sensors and to transfer them to a mobile telephone or tablet. Preferably such transfer is wireless. It is also conceivable to transfer the date to the physician's computer.

Preferably the data are processed such that they can be made available to the patient or user or physician on an app to inform them about the current success of the treatment. Thus, an app can be installed on the device in question that constitutes the receiver, whereby the app is designed such that the one or more parameter values are displayed for the user of the device. It is also conceivable that between the device constituting the receiver and the dialysis receiver there is communication such that the user of the device can control or regulate the dialysis machine and thus also the treatment.

As described above, it is also possible to use the acquired data for adjusting the prescription of a treatment, preferably in real time, i.e. during the treatment.

Furthermore the invention relates to a communication system comprising at least one dialysis machine according one of claims 1 to 11 and comprising a receiver that is designed to receive the at least one parameter value, whereby the receiver is not a component of the dialysis machine, but an external device, in particular a mobile device and in particular preferably a mobile telephone or a tablet.

Furthermore, the invention relates to a component selected from the following group: receptacle, in particular bag to receive fresh or used dialysate, tubing set to conduct a fluid, patient catheter to introduce and/or discharge dialysate into or from the patient's abdominal cavity. The at least one of the said components comprises at least one sensor designed to measure at least one parameter value of the fluid and to transmit this parameter value to at least one receiver. The said component is preferably designed with the characteristics of one of claims 1 to 11.

The present invention also relates to the use of a component according to the invention in a dialysis machine according to the invention.

As also described above, the sensor is a part of the component in question. For example, it can be arranged inside a peritoneal dialysis bag, in the inflow or outflow bag or in both. The bags can be designed such that the sensor is integrated in their wall. This applies analogously to the other components (tubing set, catheter). It is also conceivable that a sensor provided outside the components or inside the components is part of the invention.

As described above, it is conceivable that the sensor is an active or passive transponder or transmitter. It is particularly preferred when the sensor does not have its own energy supply because in that case, the component in question can be produced especially economically. It is also conceivable that the sensor is arranged in the area of a flowing fluid and that the energy required to operate the sensor is, for example, generated by a propeller wheel or the like in the flowing fluid.

The present invention relates to the dialysis machine and its components in the fluid-filled state as well as in the state in which it contains no fluid.

Further details and advantages are explained below with reference to an embodiment shown in the drawings, where

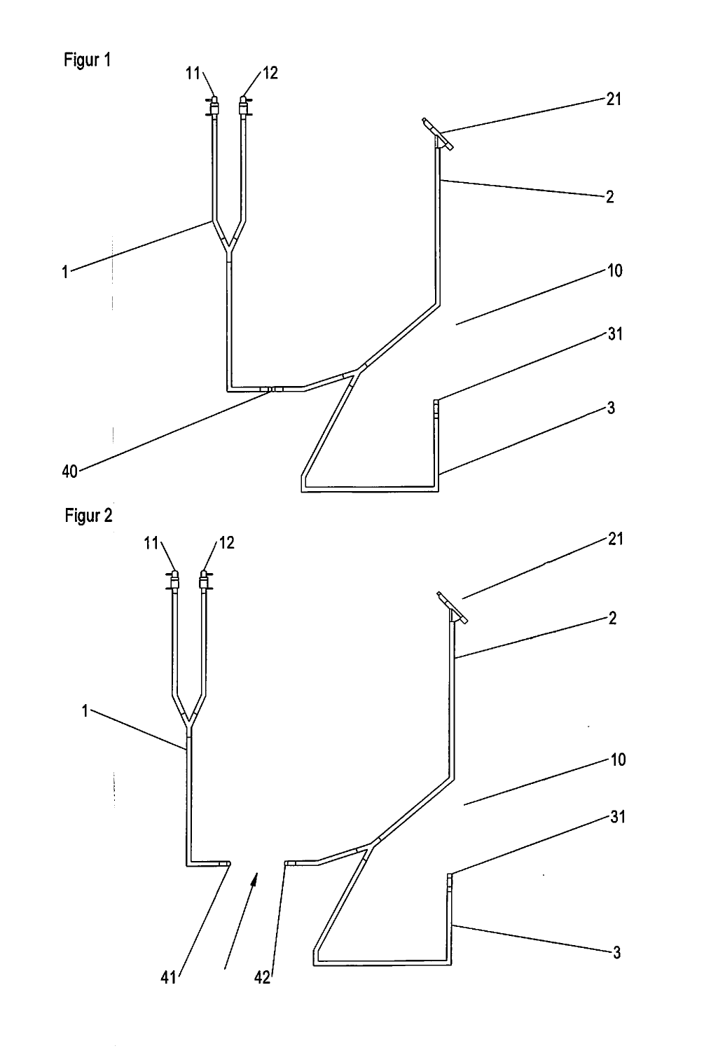

FIGS. 1-3 shows a tube before and after insertion of a sensor according to the invention.

FIG. 1 shows a tubing set (10) for use in peritoneal dialysis. The tubing set comprises a first section (1) connectable via connectors (11, 12) with bags containing fresh dialysate.

The tubing set also comprises a section (2) which is connected via connector (21) with the patient catheter, i.e. with the catheter leading into the patient's abdomen.

The tubing set also comprises a third section (3) which upon discharge of the used dialysate from the abdominal cavity is connected with a drainage bag via connector (31).

A break connector (40) such as a Luer Lock Connector is used to split section (1) of the tubing set into two parts with ends or connectors (41, 42).

FIG. 1 shows the tubing set (10) in the condition in which the dialysate is introduced from the bags (not shown) into the patient's abdominal cavity via sections (1, 2) and via the patent catheter (not shown).

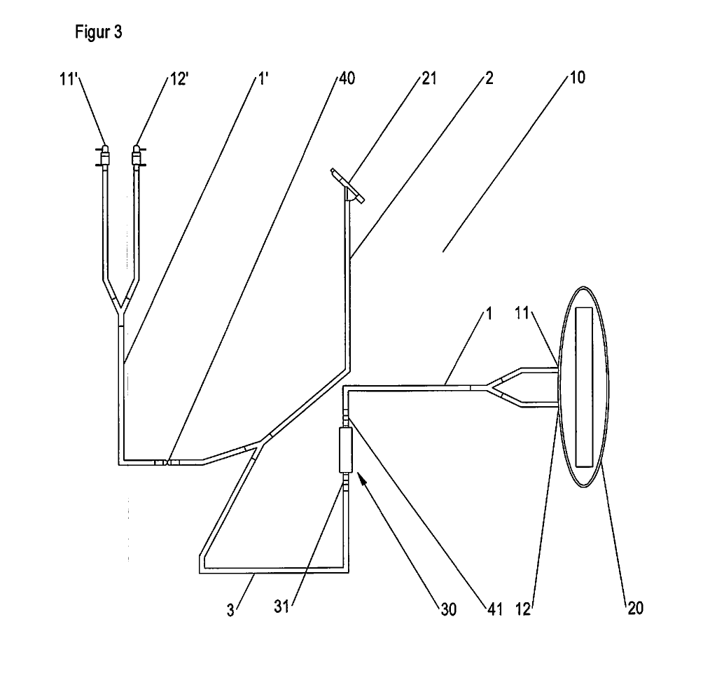

Following this introduction and after a certain dwell time in the patient's abdominal cavity, the tubing set can be split at connector (40) as indicated by an arrow in FIG. 2.

To drain the dialysate from the abdominal cavity, section (1) of the tubing set, which reaches from connectors (11, 12) to connector (41), is connected as shown in FIG. 3 with the drainage bag (20) and also with connector (31) of section (3) of the tubing set. Furthermore, a new tubing set (1′) is provided to be connected with connector (42) and is connected via connectors (11′, 12′) with bags containing fresh dialysate.

As FIG. 3 indicates, sensor (30) is provided between the end (connector 31) of section (3) and the end (connector 41) of the used section (1). Thus, due to its arrangement and as a component of the tubing set, sensor (30) is able to measure, for example, the bomposition, the metabolic breakdown products (such as urea, creatinine and electrolytes), the conductivity, the temperature, or any other parameter in the dialysate draining from the abdominal cavity. The results of these measurements can be made available to the patient and/or the physician in real time. In principle, the sensor (30) can also be arranged elsewhere, for example on the tube or on the patient connector (21).

Based on these measurements, the treatment or the prescription for the patient, such as the concentration of certain substances in the fresh dialysate or the dwell time in the abdominal cavity, can be adjusted to be optimal. Thus, the treatment to be undertaken can be adapted to the patient.

However, the invention also covers a case where the on-going treatment is not affected, but the sensor data are collected and analyzed after the treatment of the patient.

Regardless of whether a real time control or regulation of the treatment takes place or not, the dialysis machine can comprise at least one memory in which the parameter values measured by the one or more sensors can be saved in a readable format.

It is particularly advantageous when the sensor is provided with a transmitter capable of transmitting the one or more parameter values wirelessly to a mobile terminal device of the patient. For example, the patient may possess a mobile telephone or tablet or the like or also a computer which has a corresponding interface to receive the data. With these devices, the patient or also the physician can view the relevant parameter values and, where appropriate, also evaluate them.

1. A dialysis machine, in particular peritoneal dialysis machine, with one or more of the following components: at least one receptacle, in particular a bag to receive fresh or used dialysate, at least one tubing set for conducting a fluid, in particular dialysate, at least one patient catheter for the inflow and outflow of dialysate into or from the patient's abdominal cavity, characterized in that at least one of the components is provided with at least one sensor designed for measuring at least one parameter value of the fluid inside the component and also designed to transmit the measured parameter value to at least one receiver.

2. The dialysis machine according to claim 1, characterized in that the sensor is designed to transmit the at least one parameter value wirelessly to at least one receiver.

3. The dialysis machine according to claim 1, characterized in that the sensor is designed to continuously measure the at least one parameter value and/or to transmit it continuously to the at least one receiver, and/or that the sensor is designed such that the fluid flows around it or through it.

4. The dialysis machine according to claim 1, characterized in that the at least one sensor is integrated into the wall of the component or provided inside or outside on the component.

5. The dialysis machine according to claim 1, characterized in that the at least one sensor is designed as a passive transponder or as an active transponder.

6. The dialysis machine according to claim 1, characterized in that the tubing set consists of at least two sections in fluid connection with a connector and that the sensor is provided at that connector such that the sensor is in connection with both sections and is preferably arranged between these.

7. The dialysis machine according to claim 1, characterized in that the parameter is one of temperature, pressure, concentration of one or more metabolic breakdown products, of glucose level, protein level, turbidity and/or conductivity of the fluid.

8. The dialysis machine according to claim 1, characterized in that the dialysis machine comprises at least one receiver designed to receive the one or more parameter values determined by the one or more sensors and that the dialysis machine comprises at least one display and/or at least one processing unit, whereby the display unit is designed to display or otherwise communicate the one or more parameter values, and that the processing unit is designed to control and/or regulate the operation of the dialysis machine depending on the parameter values, whereby it is preferably provided that the control or regulation of the dialysis machine occurs in real time.

9. The dialysis machine according to claim 8, characterized in that the processing unit is designed to control or regulate the operation of the dialysis machine depending on the parameter values which are generated by one or more sensors not in communication with the one or more components.

10. The dialysis machine according to claim 1, characterized in that the component is a disposable.

11. The dialysis machine according to claim 1, characterized in that the receiver is not a component of the dialysis machine but of an external preferably mobile terminal device such as a smartphone or tablet.

12. A communication system comprising at least one dialysis machine according to claim 1 and comprising at least one receiver designed to receive the parameter value, whereby the receiver is not a component of the dialysis machine, but a device externally arranged for that purpose, in particular a mobile terminal device such as a smartphone or tablet.

13. A component selected from a receptacle, in particular a bag, to receive fresh or used dialysate, a tubing set to conduct a fluid, in particular dialysate, and/or a patient catheter for the inflow and/or outflow of dialysate into or from the patient's abdominal cavity, characterized in that, at least one of the said components is provided with at least one sensor designed to measure at least one parameter value of the fluid in the component and also designed to transmit the measured at least one parameter value to at least one receiver.

14. The component according to claim 13, characterized in that, this component is designed as part of a dialysis machine, in particular a peritoneal dialysis machine with one or more of the following components: at least one receptacle, in particular a bag to receive fresh or used dialysate, at least one tubing set for conducting a fluid, in particular dialysate, at least one patient catheter for the inflow and outflow of dialysate into or from the patient's abdominal cavity, characterized in that at least one of the components is provided with at least one sensor designed for measuring at least one parameter value of the fluid inside the component and also designed to transmit the measured parameter value to at least one receiver.

15. Use of a component according to claim 13 in a dialysis machine, in particular in a peritoneal dialysis machine, with one or more of the following components: at least one receptacle, in particular a bag to receive fresh or used dialysate, at least one tubing set for conducting a fluid, in particular dialysate, at least one patient catheter for the inflow and outflow of dialysate into or from the patient's abdominal cavity, characterized in that at least one of the components is provided with at least one sensor designed for measuring at least one parameter value of the fluid inside the component and also designed to transmit the measured parameter value to at least one receiver.
